(19) United States
(12) Patent Application Publication
(10) Pub. No.: US 2008/0274482 A1
(43) Pub. Date: Nov. 6, 2008

(54) BETA 1 INTEGRIN ACTIVATION AS A MARKER FOR ASTHMA

(76) Inventors: Mats W. Johansson, Madison, WI (US); William W. Busse, Madison, WI (US); Deane F. Mosher, Madison, WI (US)

Correspondence Address: WHYTE HIRSCHBOECK DUDEK S.C., 33 East Main Street, Suite 300, Madison, WI, 53703-4655, US

(21) Appl. No.: 11/420,418

(22) Filed: May 25, 2006

Related U.S. Application Data
(60) Provisional application No. 60/684,486, filed on May 25, 2005.

(51) Int. Cl. G01N 33/53; C12Q 1/02

(52) U.S. Cl. 435/7.92; 435/29; 435/7.21

(57) ABSTRACT

Methods are provided in which β₁ integrin activation on eosinophils is detected. In a first version of the method, a sample including the eosinophils is obtained from a subject. In one embodiment, the sample is whole blood. An amount of activation of β₁ integrin in the eosinophils is detected. The total number of eosinophils in the sample is quantified. In a second version of the method, a sample including eosinophils is obtained from a subject. The eosinophils are contacted with a reagent for detecting at least partially activated β₁ integrin in the eosinophils under conditions such that the reagent detects at least partially activated β1 integrin. Kits for carrying out the method are also included.

### CROSS-REFERENCE TO RELATED APPLICATIONS

This application claims priority under 35 U.S.C. §119(e) to U.S. Provisional Patent Application No. 60/684,486, filed May 25, 2005, the entirety of which is incorporated by reference herein.

### REFERENCE TO GOVERNMENT GRANT

This invention was made with United States government support awarded by the following agency: NIH HL056396. The United States has certain rights in this invention.

### BIBLIOGRAPHY

Complete bibliographic citations of the references referred to herein by authors' names can be found in the Bibliography section, immediately following the Examples. The references listed in the bibliography are incorporated by reference into the application in their entireties.

### FIELD OF THE INVENTION

The present invention relates to methods of detecting activation of β₁ integrin on eosinophils and kits for doing so.

### DESCRIPTION OF THE RELATED ART

Asthma is a chronic disease affecting over 20 million Americans. Persons with asthma have inflamed airways that are hypersensitive to physiological and environmental triggers. When triggered, asthma causes a narrowing of air passages in the lung along with the generation of sputum. Currently, a number of tests are available for diagnosing a person with asthma. However, there are limited tests for monitoring treatment efficacy of the disease and features of the disease. Collection and analysis of the sputum produced during an asthmatic attack is one method of measuring disease severity. However, this method is inconsistent and tedious. Another method for analyzing airway inflammation is through measurement of exhaled nitric oxide (NO), which is produced as a result of an increase in reactive oxygen species resulting from the body's inflammatory response. The NO exhalation test, however, is rather inaccurate; the flow rate with which the patient exhales can affect NO concentration. Also, due to the high baseline airway inflammation of a person suffering from asthma, NO levels are high in patients in which the disease is under control.

Asthma is characterized by eosinophilic inflammation. Eosinophils are a family of white blood cells that are attracted to areas where foreign substances enter the body. Eosinophils release toxic substances to kill the invaders. Recent observations have indicated that the presence of sputum eosinophils is an indicator of asthma instability or likelihood of an asthma exacerbation. Studies also suggest that the recruitment of eosinophils to the airway may be a key step in the development of an asthma exacerbation. Interaction between α₄α₁ integrin on eosinophils and vascular cell adhesion molecule-1 (VCAM-1) on cytokine-stimulated endothelium is believed to be essential for eosinophil to move from the bloodstream into tissues

Eosinophilic inflammation is a characteristic histologic feature of asthma. (Giembych M A, et al.; Busse W W, et al.)=Gibson and colleagues have shown that a gradual reduction of inhaled corticosteroids (ICS) in asthma leads to a decrease in the forced expiratory volume in one second (FEV₁), and that with the development of airflow obstruction there is an increase in circulating and sputum eosinophils. (Gibson P G, et al.) Green, et al. found that ICS treatment directed towards a reduction in sputum eosinophils improves asthma control and prevents asthma exacerbation. (Gibson P G, et al.; Green R H, et al.) These studies indicate that the presence of airway eosinophils is associated with diminished asthma control and increased risk for an exacerbation and that the recruitment of eosinophils to the airway is a key step in the development of an asthma exacerbation.

Eosinophil adhesion receptors of the integrin family and their cognate ligands in the lung are indicated as important participants in eosinophil recruitment. (Giembych M A, et al.; Seminario M C, et al.) Integrin-mediated adhesion is a function of ligand density, cell-surface integrin density, and integrin activation state. (Palecek S P, et al.) VCAM-1 is preferentially induced on the endothelial cell surface in response to mediators of T helper cell type 2 (Th2) immunity and supports specific adhesion of blood eosinophils via α₄β₁, integrin. (Masinovsky B, et al.; Giembych M A, et al; Seminario M C, et al.; Weller P F, et al.) Bronchial biopsies obtained from asthmatic subjects after a withdrawal of ICS demonstrate a higher percentage of VCAM-1-positive vessels than in the patients before withdrawal. (ten Hacken N H, et al.) Depending on its conformation and activation state, an integrin may or may not interact with a ligand and mediate cell adhesion. (Hemler M E.) Unlike many other integrin-ligand pairings, eosinophil α₄β₁ mediates adhesion to VCAM-1 in the absence of stimulation and “inside-out” activation of the integrin. (Giembych M A, et al.; Weller P F, et al.; Johansson M W, et al., 2004)

In view of the foregoing, it would be desirable to provide more reliable methods of detecting markers related to asthma control. It would also be desirable to provide kits for practicing these methods.

### SUMMARY OF THE INVENTION

The invention, which is defined by the claims set out at the end of this disclosure, is intended to solve at least some of the problems noted above. A method is provided in which β₁ integrin activation on eosinophils is detected. A sample including the eosinophils is obtained from a subject. In one embodiment, the sample is whole blood. An amount of activation of β₁ integrin in the eosinophils is detected. The total number of eosinophils in the sample is quantified.

The occurrence of the amount of activation of β₁ integrin above a minimum threshold can be used as an indicator of a decreased lung function. The occurrence of the amount of activation of β₁ integrin below a minimum threshold can be used as an indicator of an increased lung function.

The step of detecting can comprise conducting an assay to determine binding of at least partially activated β₁ integrin to a binding partner of activated β₁ integrin. The assay can be performed by flow cytometry, an ELISA assay, and an ELISA-like assay. However, other assays can also be used.

The binding partner can be an antibody that binds to at least partially activated β₁ integrin. An example of such an antibody is the monoclonal antibody N29. The total number of eosinophils in the sample can be quantified using an eosinophil peroxidase assay or other assays.

Additional steps can also be included in the method. These steps can include determining a baseline of β₁ integrin expression on the eosinophils in a baseline sample from the subject. An amount of expression of β₁ integrin on the eosinophils in an additional sample from the subject can then also be detected. The baseline of β₁ integrin expression can be compared to the amount of expression of β₁ integrin on the eosinophils in the additional sample.

Another method is provided in which β₁ integrin activation on eosinophils is detected. In the method, a sample including eosinophils is obtained from a subject. The eosinophils are contacted with a reagent for detecting at least partially activated β₁ integrin in the eosinophils under conditions such that the reagent detects at least partially activated β₁ integrin.

The level of at least partially activated β₁ integrin can be determined. The occurrence of at least partially activated β₁ integrin at a level above a minimum threshold level can be used as an indicator of a level of asthma control in the subject.

The reagent used in the method can be an antibody. When used, the antibody is capable of forming a complex with the at least partially activated β₁ integrin. The amount of complex formed can be determined and then used as a measure of the amount of at least partially activated β₁ integrin. The amount of complex determined can be used as an indicator of lung function in the subject.

A kit for determining relative activation of eosinophilic β₁ integrin in a subject is also included. The kit includes a positive control including cells with at least partially activated β₁ integrin and a negative control including cells lacking activated β₁ integrin.

The kit can also include an anti-P integrin monoclonal antibody that binds to at least partially activated β₁ integrin. Such an antibody is monoclonal antibody N29. Where N29 is used, the kit can also include a secondary antibody that binds to the N29 antibody.

The positive control can be Jurkat T cells or any other cell having at least partially activated β₁ integrin. The kit can also include another negative control that is cells lacking expressed β₁ integrin. This can be used to quantify the level of β₁ integrin expression in a sample. Both of the negative controls can be mouse cells, a knock-out cell with the β₁ subunit of integrin removed therefrom, or any other appropriate control.

### BRIEF DESCRIPTION OF THE DRAWINGS

Preferred exemplary embodiments of the invention are illustrated in the accompanying drawings, in which like reference numerals represent like parts throughout and in which:

FIG. 1 is a schematic showing the study design for Example 1. The study was a randomized, double-blind, placebo-controlled, two-period crossover inhaled corticosteroids (ICS) withdrawal trial. Patients were randomized to the order of two periods during which the known ICS was replaced by either the study ICS (sham ICS withdrawal) or placebo (true ICS withdrawal). Thus, the indicated fluticasone doses during the study periods were only known when the randomization code was broken.

FIGS. 2A-2E are graphical depictions of examples of primary flow cytometric data. Gating of eosinophils in whole blood is depicted based on side scatter (SSC) versus staining with FITC-conjugated anti-CD14 and anti-CD16 antibodies (FIG. 2A) and based on side versus forward scatter (FSC) (FIG. 2B). Fluorescence intensity of this population is shown after incubation with isotype control mouse IgG1 (FIG. 2C) or activation-sensitive anti-β₁ integrin mAb N29 (FIGS. 2D-2E) and PE-conjugated secondary antibody. Examples are shown for samples before (FIGS. 2C-2D) and after (FIG. 2E) ICS withdrawal. The isotype control was used to set the threshold for 2% positive cells (bar in FIGS. 2C-2E): In these samples there were 9% (FIG. 2D) and 86% (FIG. 2E) N29-positive blood eosinophils and the specific gMCF (geometric mean channel fluorescence) was 28 (FIG. 2D) and 507 (FIG. 2E), respectively.

FIG. 3 is a graph of Receiver-Operator Characteristic (ROC) curves for the ability of blood eosinophil expression of the epitope for activation-sensitive β₁ integrin mAb N29 (percent positive cells), FENO, or eosinophil percentage in sputum to predict FEV₁<90% of baseline. Areas under curve: N29, 0.93; FENO, 0.86; sputum eosinophils, 0.80.

FIG. 4 is a graph showing data from a study, the Severe Asthmatic Research Program (SARP) at the University of Wisconsin, comparing N29 epitope expression (y axis) and forced expiratory volume in 1 second (FEV₁) (x axis).

FIG. 5 is a graph showing β₁ integrin activation (N29 epitope expression) of purified blood eosinophils. N29 epitope expression on original eosinophil population before adhesion to VCAM-1 (normal line, to the right), N29 epitope expression on eosinophils non-adherent to VCAM-1 (heavy line, center), isotype control antibody (thin line, to the left).

It is to be understood that the invention is not limited in its application to the details of construction and the arrangement of the components set forth in the following description of embodiments of the invention or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

Abbreviations used herein include the following.

BAL=bronchoalveolar lavage; BSA=bovine serum albumin; ctrl=control; eNO=exhaled nitric oxide; eos=eosinophils; F=female; FEV₁=forced expiratory volume in ₁s; FITC=fluorescein isothiocyanate; FN=fibronectin; FSC=forward scatter; gMCF=geometric mean channel fluorescence; ICAM=intercellular adhesion molecule; ICS=inhaled corticosteroid; Ig=immunoglobulin; M=male; mAb=monoclonal antibody; Meth PC₂₀=provocative concentration of methacholine producing a fall in FEV₁ of 20%; n=number of subjects; p=probability; PBS=phosphate-buffered saline; PE=phycoerythrin; PEF=peak expiratory flow; pos=positive; pred=predicted; ppb=parts per billion; PSI=plexin, semaphorin, and integrin; ROC=Receiver-operator Characteristic; rs=Spearman rank correlation coefficient; S=sham ICS withdrawal; SARP=Severe Asthma Research Program; SD=standard deviation; SSC=side scatter; subj=subject; T=true ICS withdrawal; Th2=T helper cell type 2; VCAM-1=vascular cell adhesion molecule-1; withdr=withdrawal.

Studies have shown that asthma-induced airflow obstruction leads to an increase in the number of circulating and mucus-associated eosinophils, a specific subtype of white blood cell. Interaction between α₄β₁, integrin on eosinophils and VCAM-1 on cytokine-stimulated endothelium is considered to be essential for eosinophil invading tissue from the circulatory system. The cells must leave the circulatory system and enter the lungs in response to an asthmatic inflammation event.

To evaluate whether activation of β₁ integrins is a feature of an asthma control, eosinophil β₁ integrin expression and activation state in blood of patients undergoing controlled withdrawal of ICS were measured. Quantitation of β₁ integrin activation state was assessed by reactivity with the activation-sensitive anti-β₁ integrin monoclonal antibody (mAb) N29. Significant correlations between N29 expression and measurements of lung function determined that N29 expression is a marker of asthma.

The role of β₁ integrin activation in asthma is further supported by preliminary data from a study of patients enrolled in a Severe Asthma Research Program (SARP) at the University of Wisconsin. The SARP study is discussed below in Example 2. Briefly, preliminary data show a trend to an inverse correlation between β₁ integrin activation (N29 epitope expression) on eosinophils and lung function.

In addition, in vivo studies, described in Example 3, showed a link between N29 epitope expression and eosinophil adhesion to VCAM-1 (rₛ=0.90, p=0.08).

The inventors have found that the detection or quantitation of eosinophil β₁ integrin activation can be utilized as a biomarker predictive of a level of asthma control.

The present invention provides methods for determining disease status in a patient having asthma based on correlation between activation of β₁ integrin on eosinophils and lung function. An example of a disease status is the lung function of the patient. A binding partner, such as an antibody, may be used to detect the activation state of β₁ integrin and/or quantify the amount of β₁ integrin on eosinophils. The invention further provides kits that can be used to carry out the methods of the invention. β₁ integrin can have different activation states. First, it can be inactive. In addition, it can be partially activate with low or intermediate activity. Also, it can have high activity.

Another embodiment of the method also involves quantifying the expression of β₁ integrin on eosinophils. This can be accomplished using an MAR4 antibody, as is described below.

Obtaining a Sample Including Eosinophils from a Patient:

A sample including eosinophils, such as whole blood, lung sputum, or any other fluid containing eosinophils, can be obtained from a patient by conventional methods. The methods and kits described herein can be used on patients having a diagnosis of mild, persistent asthma or a different asthma diagnosis. The methods and kits can also be used on patients having natural fluctuations in their airway disease. Samples can be fixed after binding to an antibody, for example, as described in Example 1, such that the subsequent analysis is carried out at a later time.

In one embodiment where whole blood is used, the amount of whole blood is from about 0.3 ml to about 5 ml of whole blood. The eosinophil (eos) counts for a sample widely vary. For example, in Example 1 below, eos counts ranged from about 50,000 to 600,000/ml, with eos about 2% to about 9% of total white blood cells (WBC). The eos count for a normal, non-allergic, person (“normals”) is typically about 2% of total WBC. For an allergic asthmatic patient deliberately challenged with antigen, the eos count is up to about 19% of total WBC, which are about 15 million/ml. Typically, asthmatics without extra stimulation have an eos count of up to about 15% of WBC, or about 10 million/ml.

Analysis of a Sample for at Least Partially Activated Eosinophilic β₁ Integrin:

In one embodiment of the methods, an amount of β₁ integrin activation in the eosinophils is then detected. The occurrence of the amount of activation of β₁ integrin above a minimum threshold can be used as an indicator of a decreased lung function. The occurrence of the amount of activation of β₁ integrin below a minimum threshold can be used as an indicator of an increased lung function. That is, the level of β₁ integrin activation correlates inversely with lung function, which can be measured by, for example, FEV₁. The level of β₁ integrin activation is a predictor of lung function and asthma control.

The step of detecting can include conducting an assay to determine binding of at least partially activated β₁ integrin to a binding partner of activated β₁ integrin. The binding partner can be an antibody that binds to at least partially activated β₁ integrin. One embodiment of a method comprises detecting N29 epitopes on eosinophils utilizing an N29 monoclonal antibody. However, it should be understood that other epitopes and other monoclonal and polyclonal antibodies are included in the scope of the invention. For example, the 8E3 monoclonal antibody described in Mould et al. and the antibodies discussed hereinbelow can be used in the methods and kits of the invention.

The total number of eosinophils in the sample can be quantified using an eosinophil peroxidase assay or other assays, as is described in more detail below.

The detection of the amount of β₁ integrin activation in the eosinophils can be performed with an immunoassay. For example, in Example 1, flow cytometry is used to detect binding of the N29 antibody to the N29 epitope. Flow cytometry can also be used to detect other epitopes present on at least partially activated β₁ integrin. In another embodiment, the sample can be analyzed using an enzyme linked immunosorbant assay (ELISA) assay or an ELISA-type assay. In one embodiment of the ELISA or ELISA-type assay, a substrate, such as wells on a microtiter plate, is coated with an antibody that binds to activated β₁ integrin, such as monoclonal antibody N29. Unbound sites on the substrate can be blocked to prevent false positive results. Preferably, the substrate only binds to N29 antibody such that no cells bind directly to the substrate or to a “control” surface of the substrate with no N29 immobilized on it.

In one embodiment of the assay, the substrate possesses an inert surface. In another embodiment, the substrate is a so-called “self-assembled monolayer, as is described in, e.g., U.S. Pat. No. 6,849,321 for “Surfaces with gradients in surface topography,” U.S. Pat. No. 6,824,837 for “Liquid crystal switching mechanism,” U.S. Pat. No. 6,821,485 for “Method and structure for microfluidic flow guiding,” U.S. Pat. No. 6,797,463 for “Method and apparatus for detection of microscopic pathogens,” U.S. Pat. No. 6,746,825 for “Guided self-assembly of block copolymer films on interferometrically nanopatterned substrates,” and U.S. Pat. No. 6,486,334 for “Biomembrane mimetic surface coatings,” all of which are incorporated herein by reference in their entireties.

Self-assembling monolayers (and other substrates) can include a molecule or a group to capture the N29 antibody, such as the ligands described in Orner et al. In addition, an antibody that is engineered with a functional moiety can be utilized to bind to the capturing group in the monolayer on the surface of the substrate.

After the substrate is coated with an antibody, a sample such as blood is applied to the coated substrate. Cells within the sample having the N29 epitope at least partially exposed are captured (bound) by the antibody. The substrate is then washed to remove unbound sample. A secondary antibody can be added to bind with, for example, the antigen-antibody complex or the antibody. The secondary antibody can be conjugated to an enzyme or include a detectable label, such as phycoerythrin (PE), FITC, or the like. Where a secondary antibody is coupled to an enzyme, a development reagent, such as a substrate for the enzyme, is reacted with the enzyme to produce a detectable product, thus indicating a positive reaction. Where a secondary antibody including a detectable label is used, the label is detected, such as with a fluorometer or another suitable instrument known in the art.

The immunoassay can also be accomplished by Western blotting, 2-dimensional SDS-polyacrylamide gel electrophoresis, or other methods known in the art for detection of specific proteins. The skilled artisan will recognize that the instant invention encompasses all such well-known techniques for detection of the proteins.

In addition, the immunoassay to detect the amount of β₁ integrin activation on eosinophils can be a modification of an eosinophil peroxidase (EPO) assay, as described below. In a modified EPO assay, VCAM-1, which is normally used in the EPO assay, is replaced with the N29 monoclonal antibody.

For the eosinophil peroxide assay for the quantitation of attached eosinophils, a 96 well plate is coated in triplicate with monoclonal antibody N29 (add 100 μl per well of antibody, at an approximate concentration, e.g., 10 μg/ml). The 96 well plate can be either a tissue or a non-tissue culture treated plate. The antibody is allowed to coat for 2 hours or overnight at 37° C.

The antibody is decanted, and wells are blocked with 0.1% gelatin or FBS (heat-inactivated) for 15 minutes. Blocker is decanted, and 100 μl of whole blood (undiluted or diluted in HBSS+Ca²⁺ containing 0.2% BSA) or 10,000 eosinophils resuspended in HBSS+Ca²⁺ containing 0.2% BSA per well (100 μl) are added. The plate is incubated at 37° C., in CO₂ incubator for 60 minutes. The original eosinophil stock solution is saved for 100% control stock and is also incubated in an incubator.

During incubation, OPD substrate solution (omitting OPD itself, which is kept on ice until later in the protocol) is prepared. The OPD substrate solution contains 0.1% Triton X100 (1 ml of Triton X100 diluted with 99 ml 55 mM Tris (pH 8.0); diluted 1:10 in substrate solution), 1 mM OPD, 1 mM H₂O₂ (made fresh and diluted in 55 mM Tris and substrate solution), and enough 55 mM Tris (pH 8.0) to bring to final volume needed (typically 10 ml).

When adhesion time is up, the plate and control stock eosinophils are removed from incubator. The wash plate is washed 3× with TBS, pH 8.0. 100 μl of HBSS/0.2% BSA is added to each reaction well, and 100 μl of 100% control stock eosinophils is added to 3 additional wells. OPD (50 mM OPD: Weigh out approximately 100 mg o-phenylenediamine diHCl. Aliquot about 0.2 ml/eppendorf and freeze at −80° C.) is added to substrate solution and mixed. 100 μl of substrate solution is added to all wells and incubated 30 minutes at room temperature or long enough time for color to develop. The reaction is stopped by adding 50 μl of 4 M H₂SO₄.

The plate is read immediately on 96-well ELISA spectrophotometer at 490 nm. If necessary, the plate can be held for up to 60 minutes in dark before reading. The percent eosinophil adherence is calculated by dividing the OD₄₉₀ for experimental wells into the OD₄₉₀ for the 100% control wells and multiplying by 100.

In another embodiment of the invention, the amount of β₁ integrin activation on eosinophils is determined by contacting the eosinophils with a reagent for detecting at least partially activated β₁ integrin in the eosinophils under conditions such that the reagent detects at least partially activated β₁ integrin.

The level of at least partially activated β₁ integrin can be determined. The occurrence of at least partially activated β₁ integrin at a level above a minimum threshold level can be used as an indicator of a level of asthma control in the subject. The minimum threshold level can be determined using a Receiver-operator Characteristic (ROC) curve, as is detailed below in Example 1, or by any other method known in the art.

The reagent used in the method can be an antibody, such as the N29 monoclonal antibody or other antibodies that detect at least partially activated β₁ integrin. When such an antibody is used, the antibody is capable of forming a complex with the at least partially activated β₁ integrin. The amount of complex formed can be determined though a detectable label on the antibody or with a secondary antibody that binds to the first antibody or to the complex. The amount of complex formed is used as a measure of the amount of at least partially activated β₁ integrin. The amount of complex can be used as an indicator of lung function in the subject.

Quantifying the Total Number of Eosinophils in the Sample:

The total number of eosinophils present in a sample can be quantified. This accounts for non-eosinophils that are positive for N29, such as monocytes and neutrophils. The total number of eosinophils in a sample can be quantified using e.g., a flow cytometer and an antibody, such as anti-CD14 or CD16, that distinguishes eosinophils from other cells present in the sample.

In using an immunoassay that captures N29-positive cells, the total number of captured N29-positive eosinophils (eos) in the sample can be quantified using an eosinophil-specific assay, such as EPO assay described above, using an adhesion protein ligand that binds eosinophils, such as VCAM-1 in place of the N29 monoclonal antibody. An EPO assay is routinely used to measure adhesion of eosinophils in cell-biological experiments and can be used to detect attached eosinophils even in whole blood or other mixed population of leukocytes (i.e., the assay will determine the number of attached eos in the presence of other cells, e.g., other attached leukocytes). Measuring the attachment of eosinophils to an immobilized N29 antibody with the EPO assay is similar in principle to the EPO assay routinely used to measure eosinophil adhesion to adhesive protein ligands such as VCAM-1. (Sedgwick et al.)

The EPO adhesion assay can be used with at least about 40 μl of blood because the assay can be used to detect about 500 eos or fewer (compared to the background). The sensitivity can be increased by extending the enzyme assay longer than the standard time period. To attach 500 eos with a person with a low-end number of eos and a low-end N29 reactivity, such as 25% N29-binding eos, 40 μl of blood is required (giving 2000 eos, of which a quarter would attach to N29). If the enzyme assay is enhanced, then even less blood can be used.

Other methods of quantifying the total number of eosinophils in the sample can also be used as are known and used in the art. For example, the dye eosin can be used because eosinophils take up eosin.

The analysis of a sample for at least partially activated β₁ integrin in combination with quantifying the total number of eosinophils provides a method of determining the percent of N29 positive eosinophils in a sample.

Analysis of a Sample for Eosinophilic β₁ Integrin:

In one embodiment of the methods described above, the amount of β₁ integrin expression on eosinophils is also determined. For this, a first sample including the eosinophils is obtained from the patient. A baseline of β₁ integrin expression on the eosinophils in the first sample is determined. A second sample including the eosinophils is obtained from the patient. The second sample can be taken when, for example, a healthcare provider wants to determine asthma disease status of the patient. An amount of expression of eosinophilic β₁ integrin in the second sample is detected. The baseline is compared to the amount of expression of eosinophilic β₁ integrin in the second sample.

The steps of determining and detecting can include conducting an assay with a binding partner of β₁ integrin. The assay can be an immunoassay, as is described herein, that includes the binding partner of β₁ integrin. The immunoassay can be chosen from at least one of flow cytometry, an ELISA assay, and an ELISA-like assay. The immunoassay can also be performed with a Western blot on cell extracts or other immunoassays known in the art.

The binding partner can be an antibody that binds to β₁ integrin, such as MAR4. In Example 1, flow cytometry is used to detect binding of the MAR4 antibody binding to β₁ integrin. Other antibodies such as 4B7R or P5D2 from R&D Systems, Inc. (Minneapolis, Minn.) may be used to quantify the expression of β₁ integrin. The binding partner can also be VCAM-1, fibronectin, laminin, and other molecules that bind to β₁ integrin.

Kits to Detect Activation of β₁ Integrin:

The invention further provides kits for determining the relative activation of eosinophils. The kits can also be used for determining the level of β₁ integrin expression on eosinophils.

In one embodiment of the kit, the kit includes a positive control including cells with at least partially activated β₁ integrin. The kit can also include a negative control including cells lacking activated β₁ integrin. The kit can also include a binding partner of activated β₁ integrin, such as an anti-β₁ integrin monoclonal antibody that binds to β₁ integrin when β₁ integrin is activated. An example of an antibody that can be used in the kit is N29.

In another embodiment of the kit that can be used also for determining a relative level of expression of β₁ integrin on eosinophils, the kit includes a positive control including cells expressing β₁ integrin. The kit can also include a negative control including cells lacking expressed β₁ integrin. A binding partner of β₁ integrin can be included in the kit, such as a monoclonal antibody that binds to β₁ integrin. (Arroyo A G, et al.) An example of a monoclonal antibody useful in the kit is MAR4.

Both of the positive controls can be Jurkat T cells. Both of the negative controls can be a mouse cell. The negative controls can also be knock-out cells with β₁ subunits of integrin removed therefrom.

A secondary antibody that binds to the first antibody or to the complex of the first antibody and its antigen can be included in the kit. The secondary antibody can include a detectable label. A development reagent, such as a substrate for an antibody-linked enzyme can also be included where the secondary antibody has an enzyme conjugated to it.

The kit can be employed in laboratory settings and outside the laboratory. The kit can be adapted to be portable and for use in a patient's home. For example, the kit can be adapted to be in a format like that of home pregnancy test kits in which antibody embedded filter paper is included for use in contacting a sample from the patient. The resultant binding of the antibody with the sample could then produce a positive or negative result, or could generate a gradated result that would be compared to a representation of known results.

### EXAMPLES

The following Examples are provided for illustrative purposes only. The Examples are included herein solely to aid in a more complete understanding of the presently described invention. The Examples do not limit the scope of the invention described or claimed herein in any fashion.

### Example 1

### Methods

Subjects:

Eight asthmatic patients (Table 1) were recruited between May, 2002, and November, 2003, in the Madison (Wisconsin, USA) area and attended visits for five months until March, 2004. The patients had a diagnosis of mild, persistent asthma and an FEV₁ of ≧80% predicted. The study was approved by the University of Wisconsin-Madison Health Sciences Human Subjects Committee. Informed consent was obtained from each subject before participation, The sample size was aimed at detecting a significant change in hyperresponsiveness at a level of probability (p)≦0.05 and was based on such changes obtained in other ICS withdrawal asthma studies. (Gibson P G, et al.; Castro M, et al.)

TABLE 1
Subject characteristics

| | | | | | | Order of study |
|---|---|---|---|---|---|---|
| Sub- | | | | FEV₁ | | periods and |
| ject | Age | | PC₂₀ | | (%) | criterion* for |
| (#) | (y) | Sex | (mg/mL) | (L) | predicted) | stopping period |
| 1 | 20 | F | 16.0 | 3.27 | 100 | S (II) - T (II) |
| 2 | 21 | F | 3.1 | 3.02 | 90 | T (II) - S (II) |
| 3 | 20 | M | 3.2 | 4.71 | 98 | T (II) - S (I) |
| 4 | 19 | M | 4.2 | 5.73 | 111 | S (II) - T (I) |
| 5 | 25 | M | 1.1 | 4.27 | 94 | T (II) - S (II) |
| 6 | 21 | M | 25.0 | 4.34 | 88 | S (II) - T (II) |
| 7 | 44 | M | 1.1 | 3.64 | 80 | T (II) - S (II) |
| 8 | 24 | F | 0.9 | 4.08 | 109 | S (II) - T (II) |

Medians with 25^{th} and 75^{th} percentiles: age 21 years (20, 24); gender 3 F, 5 M; Meth PC₂₀ 3.2 mg/ml (1.1, 10); FEV₁ 4.2 1 (3.5, 4.5), 96% of predicted (89, 104).
F = female; FEV₁ = forced expiratory volume in 1 second; ICS = inhaled corticosteroid; M = male; Meth PC₂₀ = provocative concentration of methacholine producing a 20% fall in FEV₁; pred. = predicted; S = sham ICS withdrawal; T = true ICS withdrawal.
The Meth PC₂₀ and FEV₁ values are from baseline assessments at the end of the initial stabilization (i.e., before the first study period).
*Criterion I = >10% fall in FEV₁ compared to the assessments before that period, criterion II = run-in ICS discontinued for four weeks.

Withdrawal of Inhaled Corticosteroids:

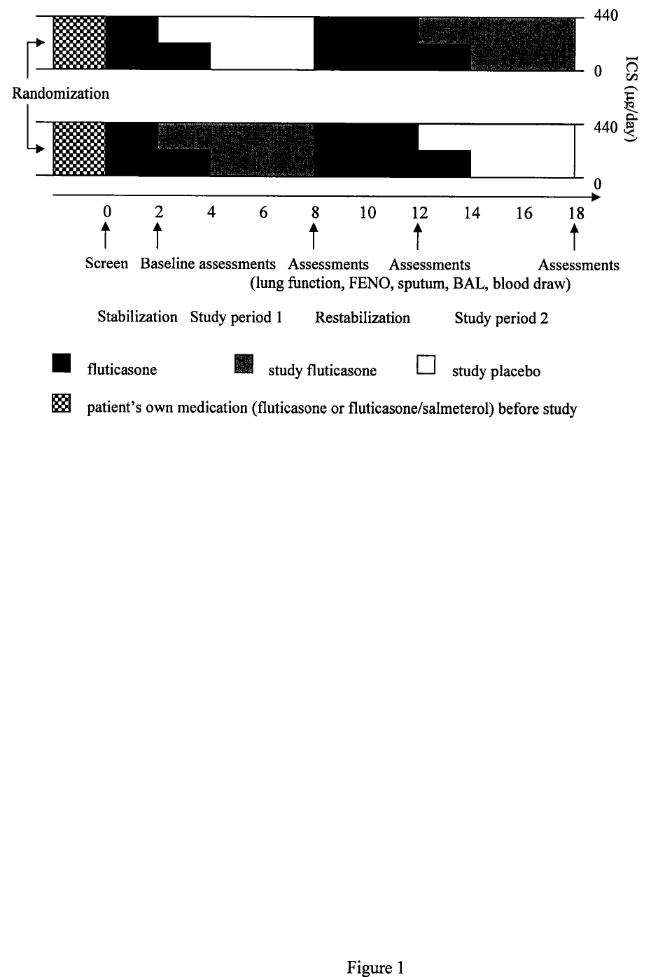

Subjects were entered on a randomized, double-blind, placebo-controlled, two-period crossover trial. Prior to entry into the study, the patients were under clinical control on ICS (fluticasone 440 μg/day or fluticasone/salmeterol (Advair) 100/50 twice per day (Table 1)) in addition to an inhaled short-acting β agonist. Subjects on combination therapy were switched to fluticasone 440 μg/day for two weeks before baseline assessments. During each study period, a subject's known ICS was reduced by 50% for two weeks and then discontinued completely. The known ICS was replaced with study ICS (sham withdrawal) or placebo (true withdrawal) (FIG. 1). The order in which the periods occurred was determined by randomization carried out by the Pharmaceutical Research Center, University of Wisconsin-Madison. A forced block design was used to ensure that equal numbers of patients were assigned to each treatment sequence. Subjects were stabilized on fluticasone, 440 μg/day, for two weeks prior to the first period and for four weeks between the two periods (FIG. 1). Each period ended when subjects (I) had a >10% fall in FEV₁ or (II) had discontinued the known ICS for four weeks.

During all periods except two, the second criterion was used to end the study period (Table 1). Complete assessments were made at the beginning and end of each study period. Assessments included measurements of airway physiology (spirometry and methacholine challenge) (Kelly E A, et al.), airway inflammation (analysis of bronchoalveolar lavage (BAL) (Liu L Y, et al., 2002; Kelly E A, et al.), sputum (Liu L Y, et al., 2000), and exhaled nitric oxide (eNO) (Strunk R C, et al.), blood draw, and rating of asthma symptom score. In addition, spirometry, check of diary cards, and assessment of possible adverse events were performed at every visit, at least once a week, throughout the study. Investigators, other study personnel, and participants were blinded to treatment sequence assignment during the study. The code was revealed to scientists once data collection and laboratory analyses were complete.

Blood, BAL, sputum, and exhaled NO:

Blood was drawn into standard lavender-top tubes, giving a final EDTA concentration of 1.8 mg/ml (BD Vacutainer Systems, Franklin Lake, N.J., USA). BAL was performed and BAL cells were recovered, cytospun, and stained for differential counts as described. (Kelly E A, et al; Liu L Y, et al., 2002) Sputum was induced, treated, cytospun, and stained for differential cell counts as described. (Liu L Y, et al., 2002) The concentration of exhaled NO was measured using the Aerocrine NIOX 2.0.3 monitoring system according to the manufacturer's instructions (Aerocrine, Stockholm, Sweden), by having subjects exhale for 10 s.

Antibodies:

Anti-β₁ mAb MAR4; anti-β₇ Fib504; anti-α₄ 9F10; anti-α₆ GoH3; phycoerythrin (PE)-conjugated goat anti-mouse and anti-rat immunoglobulin (Ig) G; fluorescein isothiocyanate (FITC)-conjugated anti-CD14 and anti-CD16; activation-sensitive anti-β₁ mAbs HUTS-21 (Luque A, et al.) and 9EG7 (Lenter, et al.); and isotype controls mouse IgG₁, K (clone A112-2) and rat IgG₂ₐ, κ (A110-2) were from BD Biosciences (San Diego, Calif., USA). Activation-sensitive anti-β₁ N29 (Wilkins J A, et al.) was from Chemicon (Temecula, Calif., USA).

Flow Cytometry:

Whole blood (100 μl) was incubated with 0.5 μg primary antibody or isotype control in 100 μl FACS buffer (phosphate-buffered saline (PBS) with 2% bovine serum albumin (BSA) and 0.2% NaN₃) for 30 min. After primary antibody incubation, samples were washed with 1 ml PBS, washed with 250 μl FACS buffer, and then resuspended in 250 μl PE-conjugated goat anti-mouse or anti-rat IgG at 20 μg/ml in FACS buffer and incubated for 30 min. Samples were washed again with PBS, resuspended in 100 μl FACS buffer with FITC-conjugated anti-CD14 (0.125 μg) and anti-CD16 (0.625 μg) and incubated for 30 min. Red blood cells were lysed by incubation with 2 ml FACS lysing solution (BD Biosciences) for 10 min, followed by centrifugation. Incubations were at room temperature until after red blood cell lysis and then at 4° C. Samples were washed with 500 μl FACS buffer, resuspended in 250 μl FACS fix (1% paraformaldehyde, 67.5 mM sodium cacodylate, 113 mM NaCl, pH 7.2), stored at 4° C. in the dark, and within one week washed with 1 ml PBS and resuspended in 250 μl FACS buffer just prior to data collection.

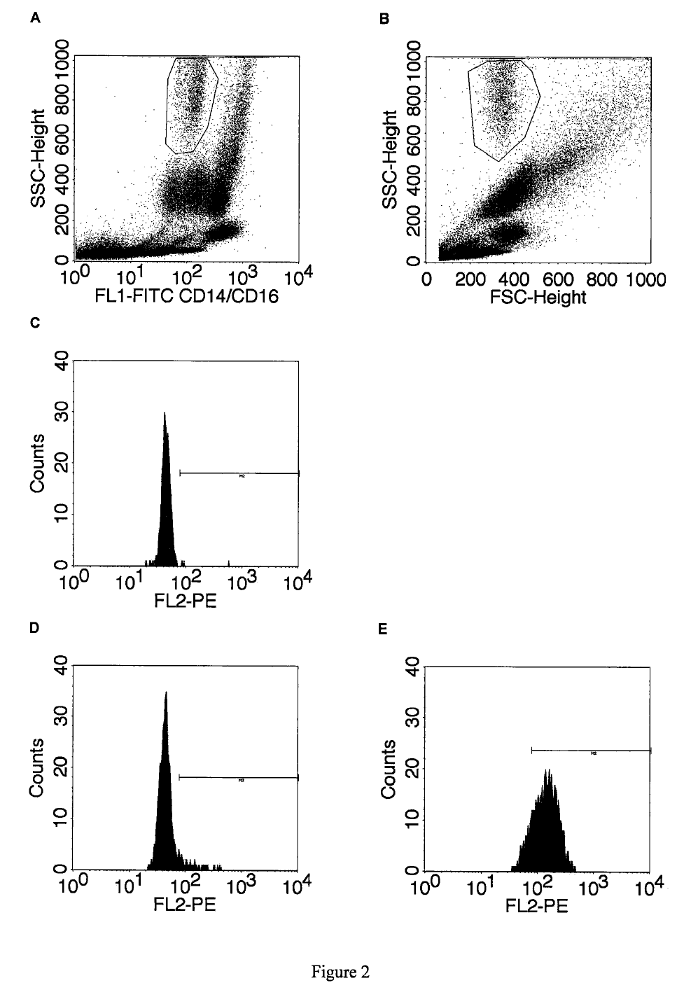

Data were collected from 30,000-170,000 events, using a FACS Calibur (BD Biosciences; available through the Flow Cytometry Facility, Comprehensive Cancer Center, University of Wisconsin-Madison, USA). Data were analyzed using Cellquest (BD Biosciences). Eosinophils were gated based both on scattering and reaction with anti-CD 14 and anti-CD 16, i.e., the cells that were analyzed for PE signal fitted two criteria for eosinophils by being gated inside both characteristic regions in a plot of side scatter versus FITC staining and a plot of side versus forward scatter (FIG. 2A,B). These criteria exclude neutrophils, monocytes, lymphocytes, and natural killer cells. The Spearman rank correlation coefficient (rs) was 0.97 (p<0.01) between the percentage of cells gated as eosinophils according to this method and the eosinophil percentage in blood after ICS withdrawal as determined by eosin staining, supporting the validity of the gating method. Data are expressed as specific geometric mean channel fluorescence (gMCF; specific gMCF=gMCF with a specific integrin mAb−gMCF with isotype control) or as the percentage of positive cells (isotype control set with a marker to 2% positive cells) (FIG. 2C-E). Of the total 32 visits, data could not be obtained from two (with mAb MAR4) and three (with mAb N29) visits, respectively. To assess reproducibility, the same blood sample was processed with the same primary antibody and analyzed by two individuals independently. Mean specific gMCF values were within 5% of each other.

Statistics:

The Mann-Whitney U test was used to compare data from groups as a whole; the Wilcoxon matched-pair signed-rank test to pairwise compare data from groups using only those patients from whom each data point was available from both groups (see Tables); and correlations were analyzed using Spearman rank correlation test. (Bluman A G.) A level of probability (p)≦0.05 was considered significant.

Receiver-operator Characteristic (ROC) curves were generated using SPSS 11.0, Chicago, Ill. Receiver-operator Characteristic (ROC) curves were run for the ability of blood eosinophil expression of the activation-sensitive β₁ integrin monoclonal antibody N29 (percent positive cells), FENO (fraction of exhaled nitric oxide), or eosinophil percentage in sputum to predict FEV less than 95% of baseline.

Results and Discussion:

When the subjects underwent true ICS withdrawal, changes in some measurements indicative of asthma control, i.e., airway sensitivity to methacholine (MethPC20) (probability [p]=0.008) and FENO (p=0.02), were significantly different than during sham withdrawal (Table 2).

TABLE 2
Results of assessments of asthma severity

| | | | AT vs | | | AS vs | ΔT vs |
|---|---|---|---|---|---|---|---|
| | True ICS withdrawal* | | BT | Sham ICS withdrawal* | | BS | ΔS |
| | Before | After | p | Before | After | p | p |
| FEV₁ (l) | 4.2 ± 0.9 | 3.9 ± 0.7 | 0.05 | 4.1 ± 0.8 | 4.0 ± 0.9 | 0.35 | 0.64 |
| (%) | 102 ± 4 | 96 ± 7 | 0.05 | 101 ± 3 | 98 ± 7 | 0.20 | 0.64 |
| PEF (l/min) | 550 ± 120 | 480 ± 140 | 0.008 | 550 ± 120 | 510 ± 110 | 0.01 | 0.11 |
| SXscore | 0.6 ± 0.7 | 2.0 ± 1.1 | 0.03 | 0.8 ± 0.7 | 1.2 ± 1.0 | 0.07 | 0.09 |
| Meth PC₂₀ | 8.7 ± 10.3 | 3.1 ± 2.9 | 0.04 | 6.8 ± 8.9 | 11.7 ± 11.2 | 0.04 | 0.008 |
| FENO (ppb) | 22 ± 14 | 56 ± 30 | 0.06 | 24 ± 15 | 19 ± 5 | 0.48 | 0.02 |
| Sp. eos (%) | 0.7 ± 1.2 | 4.8 ± 6.7 | 0.09 | 0.8 ± 1.0 | 0.8 ± 1.0 | 1.00 | 0.13 |
| BALF eos (%) | 1.1 ± 1.5 | 1.5 ± 1.9 | 0.58 | 0.6 ± 0.7 | 1.1 ± 1.4 | 0.45 | 0.79 |
| Blood eos | 0.19 ± 0.07 | 0.21 ± 0.18 | 0.74 | 0.16 ± 0.06 | 0.20 ± 0.11 | 0.29 | 0.46 |

AS vs BS = after sham (S) ICS withdrawal versus before sham ICS withdrawal; AT vs BT = after true (T) ICS withdrawal versus before true ICS withdrawal; BALF = bronchoalveolar lavage fluid; ΔT vs ΔS = change during true (T) ICS withdrawal versus change during sham (S) ICS withdrawal; eos = eosinophils; FENO = fraction of exhaled nitric oxide; FEV₁ = forced expiratory volume in 1 second, in liters and as percent of baseline; ICS = inhaled corticosteroid; Meth PC₂₀ = provocative concentration of methacholine (in mg/ml) producing a 20% fall in FEV₁; PEF = peak expiratory flow; p = probability (Wilcoxon signed-rank test (R software, Vienna, Austria, www.R-project.org)); ppb = parts per billion; SD = standard deviation; Sp. = sputum; SXscore = symptom score.
Blood eosinophils are expressed as 10⁶/ml.
The number of subjects with available data was 8 for all assessments, except 6 for BAL fluid eosinophils after true ICS withdrawal.
*Values shown are mean ± SD.

Changes in other measurements were not significantly different between the periods. However, there were significant differences in some measurements when comparing the visit after true ICS withdrawal to that before withdrawal, i.e., FEV₁ (mean after true ICS withdrawal=3.91, 96% of baseline; mean before true ICS withdrawal=4.21, 102% of baseline; p=0.05), PEF (mean after withdrawal=480 l/min, before=550 l/min, p=0.008), symptom score (mean after withdrawal=2.0, before=0.6, p=0.03), and MethPC₂₀ (mean after withdrawal=3.1 mg/ml, before=8.7 mg/ml, p=0.04) (Table 2). When comparing the visit after sham ICS withdrawal to that before sham withdrawal, mean FEV₁ was not different (p=0.35), although there were differences in individual subjects, including one who had a >10% decrease (Table 1 above).

Comparing changes in N29 and total β₁ integrin expression on blood eosinophils during the study periods, there were no significant differences (Table 3). Comparing the visit after true ICS withdrawal to that before true withdrawal (mean N29-positive cells was 50 and 28%, respectively (Table 3)), significance was not reached (p=0.23), although striking changes occurred in individual subjects (not shown).

TABLE 3
Integrin expression on blood eosinophils

| | | | AT vs | | | AS vs | ΔT vs |
|---|---|---|---|---|---|---|---|
| | True ICS withdrawal* | | BT | Sham ICS withdrawal* | | BS | ΔS |
| | Before | After | p | Before | After | p | p |
| N29 (gMCF) | 230 ± 240 | 360 ± 330 | 0.74 | 240 ± 200 | 200 ± 210 | 1.00 | 1.00 |
| (% positive) | 28 ± 18 | 50 ± 30 | 0.23 | 39 ± 22 | 37 ± 28 | 1.00 | 0.81 |
| β₁ (gMCF) | 510 ± 330 | 710 ± 460 | 0.31 | 480 ± 280 | 510 ± 150 | 1.00 | 0.84 |
| (% positive) | 64 ± 21 | 80 ± 19 | 0.15 | 70 ± 24 | 77 ± 13 | 0.60 | 0.22 |
| β₂ (gMCF) | 1000 ± 300 | 1080 ± 670 | 0.95 | 830 ± 340 | 1010 ± 220 | 0.58 | 0.38 |
| (% positive) | 88 ± 12 | 89 ± 12 | 0.48 | 88 ± 8 | 91 ± 8 | 0.61 | 0.87 |
| α_{D} (gMCF) | 470 ± 220 | 480 ± 320 | 0.84 | 270 ± 150 | 350 ± 100 | 0.44 | 0.13 |
| (% positive) | 64 ± 29 | 73 ± 23 | 0.33 | 49 ± 26 | 69 ± 13 | 0.06 | 0.31 |

AS vs BS = after sham (S) ICS withdrawal versus before sham ICS withdrawal; AT vs BT = after true (T) ICS withdrawal versus before true ICS withdrawal; ΔT vs ΔS = change during true (T) ICS withdrawal versus change during sham (S) ICS withdrawal; gMCF = geometric mean channel fluorescence; ICS = inhaled corticosteroid; p = probability (Wilcoxon signed-rank test (R software)); SD = standard deviation.
The number of subjects with available data was 8, except 7 for all antibodies after sham withdrawal and total β₁ before sham withdrawal, and 6 for N29 and α_{D} before sham withdrawal.
N29 was from Chemicon (Temecula, CA), mAbs MAR4 and L130 to total β₁ and β₂ from BD (San Diego, CA), and mAb 240I to α_{D} was a gift from ICOS (Bothell, WA).
*Values shown are mean ± SD.

Because of the considerable visit-to-visit variability in FEV₁, FENO, and sputum eosinophils and the individual variability in N29 expression, markers of loss of asthma control were correlated with N29. Correlations after true ICS withdrawal were analyzed using the Spearman rank correlation test. Across multiple study visits, p values were obtained for correlations using mixed-effect linear models of the ranked data, each including a fixed effect term for study visit and a random effect term for study subject, to account for treatment effects and within-subject correlation across visits. (Louis TA) There was an inverse correlation between percent N29-positive eosinophils and FEV₁ after ICS withdrawal (Spearman rank correlation coefficient [rₛ]=−0.74, p=0.05). When all visits were analyzed, percentage N29 expression significantly correlated with FEV₁ (p=0.01 for percent positive cells, p=0.02 for level) (Table 4). N29 correlated directly with FENO after ICS withdrawal (rₛ=0.79, p=0.03), but not when all visits were analyzed (Table 4). (Hanley J A, et al.) N29 expression did not correlate with sputum eosinophils (Table 4). Total β₁ expression did not correlate with FEV₁ but did correlate with FENO (Table 4). α_{D} expression, β₂ expression, eosinophil percentage in BAL fluid, or concentration of eosinophils in blood did not correlate with FEV₁ (not shown).

TABLE 4
Correlations between integrin expression on blood eosinophils
and lung function, fraction of exhaled nitric oxide, or the
percentage of eosinophils in sputum using all data sets

| | | | | | Sputum | |
|---|---|---|---|---|---|---|
| | FEV₁ | | FENO | | eosinophils | |
| | rₛ | p | rₛ | p | rₛ | p |
| N29 (gMCF) | −0.50 | 0.02 | 0.49 | 0.07 | 0.24 | 0.28 |
| (% positive cells) | −0.56 | 0.01 | 0.39 | 0.35 | 0.34 | 0.29 |
| Total β₁ integrin (gMCF) | −0.28 | 0.59 | 0.51 | 0.02 | 0.14 | 0.37 |
| (% positive cells) | −0.28 | 0.37 | 0.22 | 0.05 | 0.07 | 0.41 |
| Sputum eosinophils (%) | −0.18 | 0.33 | 0.34 | 0.46 | | |
| FENO | −0.27 | 0.56 | | | | |

FENO = fraction of exhaled nitric oxide; FEV₁ = forced expiratory volume in 1 second, expressed as percent of baseline; gMCF = geometric mean channel fluorescence; N29 = expression of the epitope for the activation-sensitive β₁ integrin monoclonal antibody N29; p = probability (repeated measures rank regression test (Louis TA) (R software); rₛ = Spearman rank correlation coefficient (Prism 3.0, GraphPad, San Diego, CA).
Correlations are among data from all visits (before inhaled corticosteroid (ICS) withdrawal, after ICS withdrawal, before sham ICS withdrawal, and after sham withdrawal), where data were available. The number of visits with available data was 29 for measurement of N29 expression, 30 for total β₁ integrin expression, and 32 for sputum eosinophils and FENO.
N29 (Wilkins J A, et al.) was from Chemicon (Temecula, CA) and mAb MAR4 to total β₁ from BD (San Diego, CA).

The correlations with FEV₁ raise the possibility that N29 expression on eosinophils in peripheral blood is a possible marker for the level of asthma activity or control. Although N29 correlated with FEV₁ regardless of whether only the visits after true ICS withdrawal or all visits were analyzed, it is not possible to conclude from the current study whether it correlates with the severity of the underlying asthma and/or with the magnitude of the response to ICS withdrawal. A larger study of asthmatics of a range of severities on or off ICS will be required to make such conclusions.

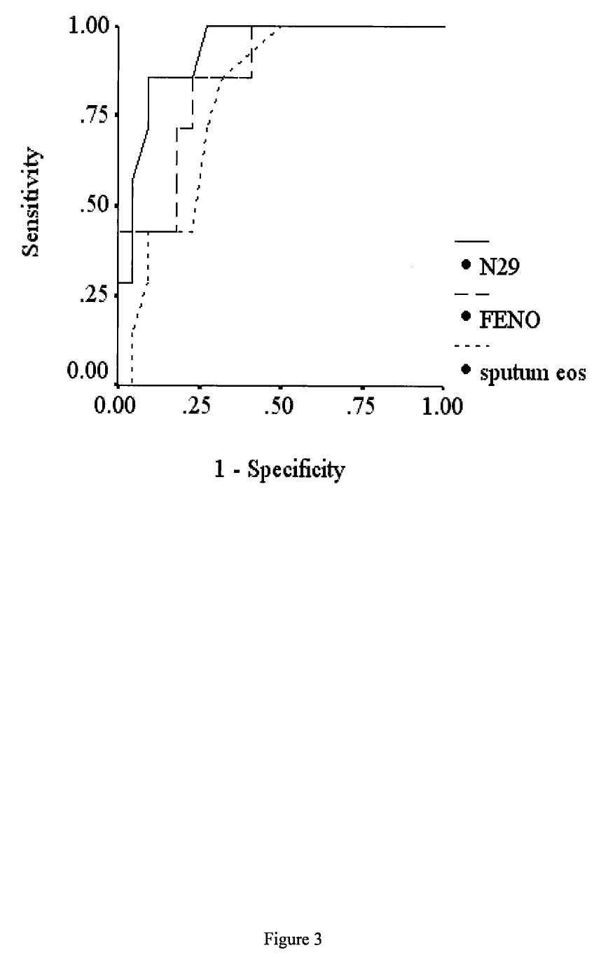

N29 expression correlated better with FEV₁ (p=0.01) than did percentage of eosinophils in sputum (p=0.33) or FENO (p=0.56), two existing biomarkers of asthma control. (Bochner B S, et al.) We carried out receiver-operator characteristic (ROC) curve analyses on the ability of N29, FENO, and sputum eosinophils to predict FEV₁<95% of baseline. (Hanley J A, et al.) N29 performed well (area under curve [AUC]=0.93) and better than did FENO (AUC=0.86) and sputum eosinophils (AUC=0.80) (FIG. 3).

### Example 2

### Materials and Methods

### 1. Subjects:

The Severe Asthma Research Program (SARP) study enrolls four groups of subjects as follows: 1) subjects with American Thoracic Society (ATS)-defined severe asthma; 2) subjects with severe, reversible asthma (same degree of airflow obstruction as group 1 but with reversible obstruction following an inhaled β agonist); 3) subjects with mild/moderate asthma, and 4) normal controls. (Proceedings of the ATS Workshop on Refractory Asthma) Informed consent is obtained from each subject before participation. Subjects are characterized by lung functions, including spirometry to determine forced expiratory volume in 1 second (FEV₁); medication use; and evaluation of sputum and airway inflammation. Measurements of lung function are performed according to ATS standards as described and done previously. (Kelly E A, et al.; Johansson M W, et al., in press). FEV₁ is measured at the same visits as when blood is drawn and is expressed as percentage of predicted value.

Approval was obtained from the University of Wisconsin Institutional Review Board for additional blood to be drawn from the SARP study for our purposes, and a change in the SARP protocol was approved to incorporate this. In addition to the consent to participate in the SARP study, informed consent is obtained from subjects who agree to have additional blood drawn for our study on β1 integrin activation.

### 2. Flow Cytometric Analysis of N29 Epitope Expression on Eosinophils in Whole Blood:

Expression of the epitope for the activation-sensitive anti-β1 integrin monoclonal antibody N29 is measured on eosinophils in whole blood by flow cytometric analysis as described and as follows: N29 is obtained from Chemicon (Temecula, Calif.). Isotype control mouse immunoglobulin (Ig) G₁, κ (clone A112-2), phycoerythrin (PE)-conjugated goat anti-mouse IgG, fluorescein isothiocyanate (FITC)-conjugated anti-CD14 and anti-CD16 are from BD Biosciences (San Diego, Calif.). (Johansson M W, et al., in press; Wilkins J A, et al.)

The additional blood for this purpose is drawn into CTAD tubes, containing citrate, theophylline, adenosine, and dipyridamole (BD Vacutainer Systems, Franklin Lake, N.J.), to minimize platelet activation. Whole blood (100 μl) is incubated with 0.5 μg primary antibody or isotype control in 100 μl FACS buffer (phosphate-buffered saline (PBS) with 2% bovine serum albumin and 0.2% NaN₃) for 30 min. After primary antibody incubation, samples are washed with 1 ml PBS, washed with 250 μl FACS buffer, and then resuspended in 250 μl PE-conjugated goat anti-mouse IgG at 2 μg/ml in FACS buffer and incubated for 30 min. Samples are washed again with PBS, resuspended in 100 μl FACS buffer with FITC-conjugated anti-CD14 (0.125 μg) and anti-CD16 (0.625 μg) and incubated for 30 min. Red blood cells are lysed by incubation with 2 ml FACS lysing solution (BD Biosciences) for 10 min, followed by centrifugation. Incubations are at room temperature. Samples are washed with 500 μl FACS buffer, resuspended in 250 μl FACS fix (1% paraformaldehyde, 67.5 mM sodium cacodylate, 113 mM NaCl, pH 7.2), stored at 4° C. in the dark, and washed with 1 ml PBS and resuspended in 250 μl FACS buffer just prior to data collection.

Data are collected from 30,000-170,000 events, using a FACS Scan or Calibur (BD Biosciences; available through the Flow Cytometry Facility, Comprehensive Cancer Center, University of Wisconsin-Madison) and Cellquest (BD Biosciences) software. At each time of data collection, “rainbow beads” (Spherotech) are first run at setup in order to calibrate the instrument at a standardized level of sensitivity and optimize data comparisons among visits.

Data are analyzed and post-data collection compensation for possible overlap between fluorochromes is performed, computed using matrix algebra by FlowJo (Tree Star). This removes any degree of subjectivity introduced by the necessary manual compensation performed at setup and thus optimizes comparisons of data among different visits. Eosinophils are gated based both on scattering and reaction with anti-CD14 and anti-CD16, i.e., the cells that are analyzed for PE signal fit two criteria for eosinophils by being gated inside both characteristic regions in a plot of side scatter versus FITC staining and a plot of side versus forward scatter. These criteria exclude neutrophils, monocytes, lymphocytes, and natural killer cells. Data are expressed as specific geometric mean channel fluorescence (gMCF; specific gMCF=gMCF with a specific integrin mAb−gMCF with isotype control).

### 3. Statistics:

The Spearman rank correlation test (Prism 3.0 software, GraphPad, San Diego, Calif.) is used to analyze the correlation between N29 epitope expression and FEV₁.

### Results:

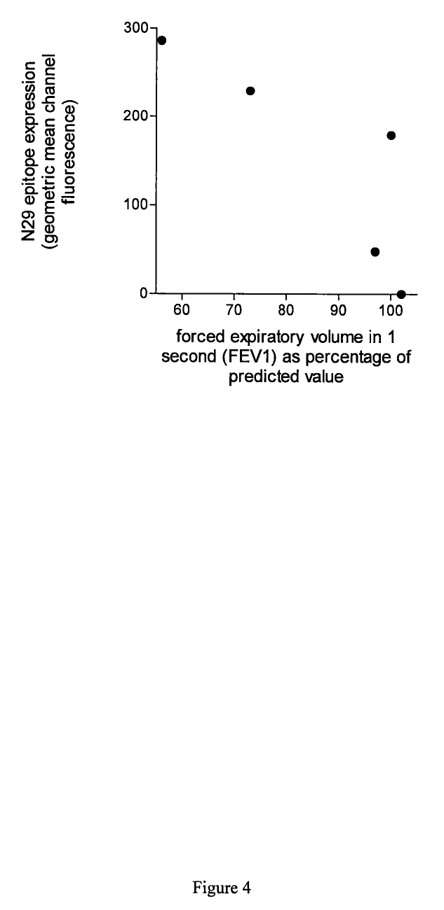

Five samples from the SARP study have been analyzed. The subjects analyzed have varying asthma severity from mild to severe and varying lung function. The preliminary data show a trend to an inverse correlation between beta1 integrin activation (N29 epitope expression) on eosinophils and lung function (FIG. 4). Expression of the epitope for the activation-sensitive anti-β₁ integrin monoclonal antibody N29 was measured on eosinophils by flow cytometric analysis as described in whole blood samples obtained from visits by SARP study patients. (Johansson M W, et al., in press) N29 epitope expression is expressed as geometric mean channel fluorescence. Lung function (forced expiratory volume in 1 second (FEV₁)) was measured at the UW Hospital at the same visits and is expressed as percentage of predicted value. rₛ (Spearman rank correlation coefficient)=0.90, p (probability)=0.08. These preliminary data indicate the possibility that β₁ integrin activation/N29 epitope expression will be higher in severe asthmatics than in mild/moderate asthmatics, and correlate inversely with lung function in this heterogeneous group of subjects.

### Example 3

### 1. In Vitro Analysis of Blood Eosinophils

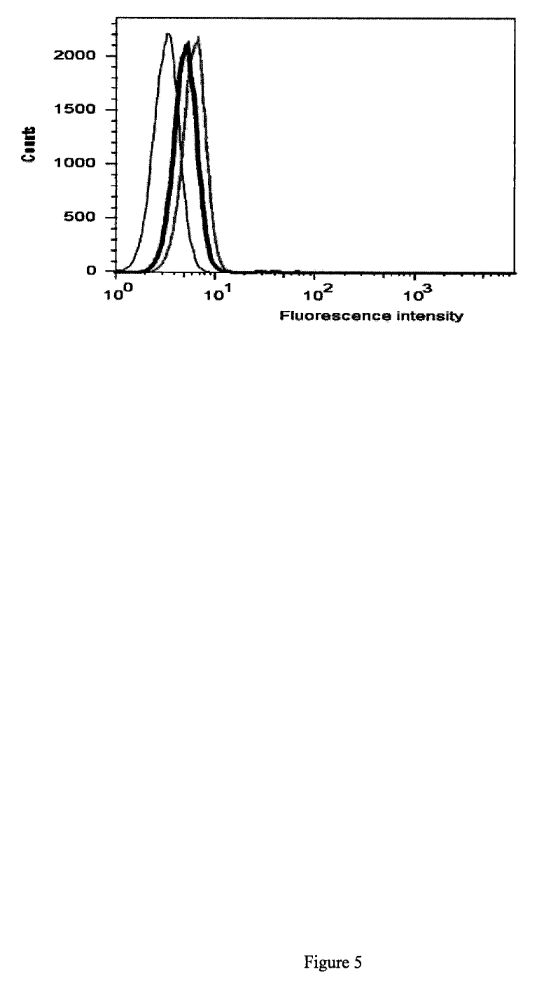

Purified blood eosinophils were plated on a substrate coated with VCAM-1, the major target of eosinophil α₄β₁ integrin. We found that the proportion of eosinophils that did not adhere to VCAM-1 had decreased β₁ integrin activation/N29 epitope expression compared to the original eosinophil population and that the highest expressing cells from the original population were lost, i.e., had adhered to VCAM-1 (FIG. 5). These results show a link between N29 epitope expression and eosinophil adhesion to VCAM-1.

In contrast to the adherent eosinophils, the vast majority of eosinophils that did not adhere to a control protein, gelatin, had the same N29 epitope expression level as the original population (not shown). These results indicate that VCAM-1 preferentially supports adhesion of those eosinophils with the highest N29 epitope expression and indicate a link between a modestly elevated N29 epitope expression and the eosinophil's capacity to adhere to VCAM-1. Such a finding provides an experimental rationale for the relevance of the N29 epitope expression on blood eosinophils to asthma: VCAM-1 expression on endothelium appears in asthma when the endothelium is activated in response to cytokines, and eosinophil adhesion to VCAM-1 is physiologically relevant and an essential step in the recruitment of eosinophils to the airway, which likely is a key step in the development of bronchial inflammation and asthma exacerbation.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention is not intended to be limited to the preferred embodiments described above, but rather is intended to be limited only by the claims set out below.

### BIBLIOGRAPHY

Arroyo A G, Taverna D, Whittaker C A, et al. In vivo roles of integrins during leukocyte development and traffic: insights from the analysis of mice chimeric for alpha 5, alpha v, and alpha 4 integrins. J Immunol 2000; 165:4667-75
Bluman A G. Elementary statistics: A step by step approach. Dubuque, Iowa: Wm. C. Brown, 1992
Bochner B S, Busse W W. Allergy and asthma. J Allergy Clin Immunol 2005; 115; 953-9
Busse W W, Lemanske R F, Jr. Asthma. N Engl J Med 2001; 344:350-62.
Castro M, Bloch S R, Jenkerson M V, et al. Asthma exacerbations after glucocorticoid withdrawal reflects T cell recruitment to the airway. Am J Respir Crit. Care Med 2004; 169:842-9)
Giembych M A, Lindsay M A. Pharmacology of the eosinophil. Pharmacol Rev 1999; 51:213-339
Gibson P G, Wong B J, Hepperle M J, et al. A research method to induce and examine a mild exacerbation of asthma by withdrawal of inhaled corticosteroid. Clin Exp Allergy 1992; 22:525-32
Green R H, Brightling C E, McKenna S, et al. Asthma exacerbations and sputum eosinophil counts: a randomised controlled trial. Lancet 2002; 360:1715-21.
Hanley J A, McNeil B J. A method of comparing the areas under receiver operating characteristic curves derived from the same cases. Radiology 1983; 160:93-9
Hemler M E. Integrins. In: Kreis T, Vale R, eds. Guidebook to the extracellular matrix, anchor and adhesion proteins, 2nd ed. New York: Oxford University Press, 1999:196-212.
Johansson M W, Lye M H, Barthel S R, Duffy A K, Annis D S, Mosher D F. Eosinophils adhere to vascular cell adhesion molecule-1 via podosomes. Am J Respir Cell Mol Biol 2004; 31:413-22.
Johansson M W, Barthel S R, Swenson C A, Evans M D, Jarjour N N, Mosher D F, Busse W W. Eosinophil β₁ integrin activation state correlates with asthma activity in a blinded study of inhaled corticosteroid withdrawal. J Allergy Clin Immunol 2006; in press
Kelly E A, Busse W W, Jarjour N N. Inhaled budesonide decreases airway inflammatory response to allergen. Am J Respir Crit. Care Med 2000; 162:883-90
Lenter M, Uhlig H, Hamann A, Jenö P, Imhof B, Vestweber D. A monoclonal antibody against an activation epitope on mouse integrin chain beta I blocks adhesion of lymphocytes to the endothelial integrin alpha 6 beta 1. Proc Natl Acad Sci USA 1993; 90:9051-5
Lindsay M A. Pharmacology of the eosinophil. Pharmacol Rev 1999; 51:213-339
Liu L Y, Sedgwick J B, Bates M E, et al. Decreased expression of membrane IL-5 receptor alpha on human eosinophils: I. Loss of membrane IL-5 receptor alpha on airway eosinophils and increased soluble IL-5 receptor alpha in the airway after allergen challenge. J Immunol 2002; 169:6452-8
Liu L Y, Swenson C A, Kelly E A, Kita H, Busse W W. The relationship of sputum eosinophilia and sputum cell generation of IL-5. J Allergy Clin Immunol 2000; 106:1063-9
Louis T A. General methods for analysing repeated measures. Stat Med 1988; 7:29-45
Luque A, Gómez M, Puzon W, Takada Y, Sanchez-Madrid F, Cabanas C. Activated conformations of very late activation integrins detected by a group of antibodies (HUTS) specific for a novel regulatory region (355-425) of the common beta 1 chain. J Biol Chem 1996; 271:11067-75
Masinovsky B, Urdal D, Gallatin WM. IL-4 acts synergistically with IL-1 beta to promote lymphocyte adhesion to microvascular endothelium by induction of vascular cell adhesion molecule-1. J Immunol 1990; 145:2886-95
Mould A P, Travis M A, Barton S J, Hamilton J A, Askari J A, Craig S E, Macdonald P R, Kammerer R A, Buckley P A, Humphries M J. Evidence that monoclonal antibodies directed against the integrin beta subunit plexin/semaphorin/integrin domain stimulate function by inducing receptor extension. J. Biol. Chem. 2005; 280:4238-46
Orner B P, Derda R, Lewis R L, Thomson J A, Kiessling L L. Arrays for the combinatorial exploration of cell adhesion. 2004, J. Am. Chem. Soc. 126:10808-9
Palecek S P, Loftus J C, Ginsberg M H, Lauffenburger D A, Horwitz A F. Integrin-ligand binding properties govern cell migration speed through cell-substratum adhesiveness. Nature 1997; 385:537-40.
Proceedings of the ATS workshop on refractory asthma: current understanding, recommendations, and unanswered questions. American Thoracic Society. Am J Respir Crit Care Med 2000; 162:2341-51
R software, Vienna, Austria, www.R-project.org
Sedgwick J B, Calhoun W J, Vrtis R F, Bates M E, McAllister P K, Busse W W. Comparison of airway and blood eosinophil function after in vivo antigen challenge. 1992. J. Immunol. 149:3710-18.
Seminario M C, Bochner B S. Expression and function of beta 1 integrins on human eosinophils. Mem Inst Oswaldo Cruz 1997; 92(Suppl 2):157-64.
Strunk R C, Szefler S J, Phillips B R, et al. Relationship of exhaled nitric oxide to clinical and inflammatory markers of persistent asthma in children. J Allergy Clin Immunol 2003; 112:883-92
ten Hacken N H, Postma D S, Bosma F, et al. Vascular adhesion molecules in nocturnal asthma: a possible role for VCAM-1 in ongoing airway wall inflammation. Clin Exp Allergy 1998; 28: 1518-25.
Weller P F, Rand T H, Goelz S E, Chi-Rosso G, Lobb R R. Human eosinophil adherence to vascular endothelium mediated by binding to vascular cell adhesion molecule 1 and endothelial leukocyte adhesion molecule 1. Proc Natl Acad Sci USA 1991; 88:7430-3
Wilkins J A, Li A, Ni H, Stupack D G, Shen C. Control of beta1 integrin function. Localization of stimulatory epitopes. J Biol Chem 1996; 271:3046-51

What is claimed is:

1. A method of detecting β₁ integrin activation on eosinophils, the method comprising:
(a) obtaining a sample including the eosinophils from a subject;
(b) detecting an amount of β₁ integrin activation in the eosinophils; and
(c) quantifying the total number of eosinophils in the sample.

2. The method of claim 1, wherein occurrence of the amount of β₁ integrin activation above a minimum threshold is indicative of a decreased lung function in the subject and occurrence of the amount of β₁ integrin activation below a minimum threshold is indicative of increased lung function in the subject.

3. The method of claim 1, wherein the step of detecting comprises conducting an assay to determine binding of at least partially activated β₁ integrin to a binding partner of activated β₁ integrin.

4. The method of claim 3, wherein the step of conducting an assay is chosen from at least one of flow cytometry, an ELISA assay, and an ELISA-like assay.

5. The method of claim 3, wherein the binding partner is an antibody that binds to at least partially activated β₁ integrin.

6. The method of claim 5, wherein the antibody comprises N29.

7. A method of claim 1, further comprising:
(a) determining a baseline of β₁ integrin expression on the eosinophils in a baseline sample from the subject;
(b) detecting an amount of expression of β₁ integrin on the eosinophils in an additional sample from the subject; and
(c) comparing the baseline of β₁ integrin expression to the amount of expression of β₁ integrin on the eosinophils in the additional sample.

8. A method of detecting β₁ integrin activation on eosinophils, the method comprising:
(a) obtaining a sample including eosinophils from a subject; and
(b) contacting the eosinophils with a reagent for detecting at least partially activated β₁ integrin in the eosinophils under conditions such that the reagent detects at least partially activated β₁ integrin.

9. The method of claim 8, wherein the level of at least partially activated β₁ integrin is determined.

10. The method of claim 8, wherein the occurrence of at least partially activated β₁ integrin at a level above a minimum threshold level is indicative of a level of asthma control in the subject.

11. The method of claim 8, wherein the reagent is an antibody.

12. The method if claim 11, wherein the antibody is capable of forming a complex with the at least partially activated β₁ integrin, and further comprising determining the amount of complex formed as a measure of the amount of at least partially activated β₁ integrin, wherein the amount of complex determined is indicative of a lung function in the subject.

13. A kit for determining relative activation of eosinophilic β₁ integrin in a subject, the kit comprising:
(a) a positive control comprising cells with at least partially activated β₁ integrin; and
(b) a negative control comprising cells lacking activated β₁ integrin.

14. The kit of claim 13, further comprising an anti-β₁ integrin monoclonal antibody that binds to at least partially activated β₁ integrin.

15. The kit of claim 14, wherein the anti-β₁ integrin monoclonal antibody comprises N29.

16. The kit of claim 13, wherein the positive control comprises Jurkat T cells.

17. The kit of claim 13, comprising another negative control including cells lacking expressed β₁ integrin.

18. The kit of claim 17, wherein the negative controls comprise mouse cells.

19. The kit of claim 17, wherein the negative controls comprise a knock-out cell with β₁ subunit of integrin removed therefrom.

20. The kit of claim 13, further comprising:
(a) an anti-β₁ integrin monoclonal antibody comprising N29; and
(b) a secondary antibody that binds to the N29 antibody.
